(19) United States
(12) Patent Application Publication
(10) Pub. No.: US 2005/0246037 A1
(43) Pub. Date: Nov. 3, 2005

(54) Partial esophageal obstruction to limit food intake for treatment of obesity

(75) Inventor: Warren L. Starkebaum, Plymouth, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/837,074

(22) Filed: Apr. 30, 2004

(51) Int. Cl. A61F 2/04

(52) U.S. Cl. 623/23.64; 606/157

(57) ABSTRACT

Medical devices and methods for treatment for obesity forming a partial obstruction in the esophagus to limit food intake. The partial obstruction limits the rate of food intake by a patient, discouraging the patient from quickly consuming an excessive amount of food at one time. In particular, the partial obstruction physically restricts the passage of food, and can cause patient discomfort when an excessive amount of food is consumed.

### FIELD OF THE INVENTION

The invention relates to medical devices and methods for treatment of obesity.

### BACKGROUND

Obesity is a major health concern in the United States and other countries. A significant portion of the population is overweight with the number increasing every year. Obesity is one of the leading causes of preventable death. Obesity is associated with several co-morbidities that affect almost every body system. Some of these co-morbidities include: hypertension, heart disease, stroke, high cholesterol, diabetes, coronary disease, breathing disorders, sleep apnea, cancer, gallstones, and musculoskeletal problems. An obese patient is also at increased risk of developing Type II diabetes.

Multiple factors contribute to obesity, including physical inactivity and overeating. A variety of medical approaches have been devised for treatment of obesity. Existing therapies include diet, exercise, appetite suppressive drugs, metabolism enhancing drugs, surgical restriction of the gastric tract, and surgical modification of the gastric tract. In general, surgery is reserved for patients in whom conservative measures, such as monitoring caloric intake or controlling appetite with appetite suppressants, have failed. In addition, surgery is generally reserved for patients who are seriously, and sometimes morbidly, overweight.

There have been many surgical approaches to obesity. For example, some patients have received implantation of one or more bulking prostheses to reduce stomach volume. A bulking prosthesis resides within the stomach and limits the amount of food the stomach can hold, theoretically causing the patient to feel a sensation of satiety. U.S. Published Patent Application No. 20030040804 to Stack et al., for example, describes a tubular prosthesis that is designed to induce sensations of satiety within a patient.

Another approach is restrictive surgery, which surgically makes the stomach smaller by removing or closing a section of the stomach. This procedure also reduces the amount of food the stomach can hold, causing the patient to feel full. U.S. Published Patent Application No. 20020183768 to Deem et al., which describes a recent proposal for treating obesity, discloses various techniques for reducing the size of the stomach pouch to limit caloric intake, as well as to provide an earlier feeling of satiety.

Another surgical procedure to treat obesity is the gastric bypass procedure. In the gastric bypass procedure, the surgeon creates a small stomach pouch to restrict food intake and constructs a bypass of the duodenum and other segments of the small intestine. This procedure limits the amount of food that is completely digested or absorbed.

Surgical procedures for treatment of obesity, such as those described above, tend to be highly invasive, and each form of surgery may involve complications. Restrictive surgery may entail a risk of vomiting, for example, and gastric bypass surgery may result in unpleasant consequences known as “dumping syndrome.”

Another surgical technique is described in U.S. Pat. No. 6,427,089 to Knowlton. In particular, Knowlton describes a surgical technique for causing a contraction or reduction in the volume of the stomach by the delivery of thermal energy to the stomach wall. According to Knowlton, the technique relies on a microwave device to heat a submucosal layer of tissue within the stomach wall without thermal damage of the mucosa of the stomach. A resulting thermal lesion causes contraction of the preexisting collagen matrix of the stomach wall.

A further technique is described in PCT Publication No. WO 00/69376 to Edwards in which nerves responsible for the sensations of hunger are ablated by applying energy to the interior mucosal lining of the stomach. The mucosal lining of the stomach, which is responsible for protecting the stomach tissue and producing stomach acid necessary for digestion, is ablated along with the specified nerves.

U.S. Pat. No. 6,540,789 to Silverman describes a technique for treatment of obesity involving introduction of an implant material into the stomach wall in the vicinity of the pyloric sphincter to inhibit emptying of the stomach. Silverman also describes introduction of an implant material to reduce distensibility and contractility of the stomach.

Table 1 below lists documents that disclose techniques for treatment of obesity.

TABLE 1

| Patent Number | Inventors | Title |
|---|---|---|
| 20020183768 | Deem et al. | Obesity treatment tools and methods |
| 20030040804 | Stack et al. | Satiation devices and methods |
| WO/0187335 | Uhiman | Method for selectively inhibiting |
| | et al. | ghrelin action |
| 6,427,089 | Knowlton | Stomach treatment apparatus and |
| | | method |
| 5,782,798 | Rise | Techniques for treating eating disorders |
| | | by brain stimulation and drug infusion |
| WO 00/69376 | Edwards | Surgical weight control device |
| 5,423,872 | Cigaina | Process and device for treating obesity |
| | | and syndromes related to motor |
| | | disorders of the stomach of a patient |
| 5,188,104 | Wemicke | Treatment of eating disorders by |
| | et al. | nerve stimulation |
| 6,540,789 | Silverman | Method of treating morbid obesity |
| 2003/0109935 A1 | Geitz | Intragastric prosthesis for treatment |
| | | of morbid obesity |
| 2003/0109931 A1 | Geitz | Intragastric stent for duodenum |
| | | bypass |

All documents listed in Table 1 above are hereby incorporated by reference herein in their respective entireties. As those of ordinary skill in the art will appreciate readily upon reading the Summary of the Invention, Detailed Description of the Preferred Embodiments and claims set forth below, many of the devices and methods disclosed in the patents of Table 1 may be modified advantageously by using the techniques of the present invention.

### SUMMARY

In general, the invention is directed to medical devices and methods for treatment of obesity. The invention provides methods and devices for forming a partial obstruction in the esophagus to limit food intake and thereby treat obesity. The partial obstruction limits the rate of food intake by a patient, discouraging the patient from quickly consuming an excessive amount of food at one time.

Various embodiments of the present invention provide solutions to one or more problems existing in the prior art with respect to prior techniques for treatment of obesity. The problems include, for example, the limited efficacy and side effects of conventional appetite suppressant medications, and the need for potential repeated dosages of such medications by the patient. Additional problems relate to the general undesirability, invasiveness, infection risk, and recovery time associated with conventional surgical techniques for treatment of obesity, such as gastric reduction and bypass surgery, and other techniques for altering the shape or size of the stomach. Side effects of some invasive procedures, such as vomiting and “dumping syndrome,” are also undesirable. Further problems relate to the need for chronic implant of prostheses within the interior of the stomach to induce satiety, and the limited effectiveness of such prostheses.

Various embodiments of the present invention are capable of solving at least some of the foregoing problems. For example, a medical device and method in accordance with the invention can provide a treatment for obesity that presents reduced side effects, relative to administration of conventional appetite suppressant medications. In some embodiments, the invention is capable of endoscopic deployment via the esophagus, and can thereby avoid the need for invasive surgical procedures. In this manner, the invention may also be capable of avoiding substantial reconstruction of the stomach, and offers reduced damage, recovery time, and side effects to the patient. Moreover, the invention does not require the presence of a chronically implanted prosthesis within the interior of the stomach.

Various embodiments of the invention may possess one or more features to solve the aforementioned problems in the existing art. In some embodiments, a method for treatment of obesity comprises implanting one or more bulking devices in a wall of an esophagus of a patient. The bulking devices are implanted at a position above the lower esophageal sphincter (LES). The implanted bulking devices are sized and shaped to extend the esophageal wall inward to create a partial obstruction of the esophagus. The partial obstruction permits food to pass downward through the esophagus, but at a limited rate relative to an unobstructed esophagus.

When an excessive amount of food is consumed in a short period of time, the partial obstruction limits the rate at which the food may pass through the esophagus and downward to the LES and stomach. In this case, the partial obstruction causes discomfort for the patient, and discourages the patient from consuming additional food until the partial obstruction has cleared. The bulking devices may be surgically implanted, e.g., by laparoscopic techniques, or endoscopically implanted via an esophagus of the patient.

The bulking devices may be expandable following implantation. In some embodiments, the bulking device includes a solid, hydrogel material that is expandable. In particular, the hydrogel material may be at least partially dehydrated prior to implantation, and then expand substantially due to rehydration following implantation. For example, the hydrogel material may be expandable from a pre-implantation volume of less than approximately 100 mm³ to a post-implantation volume of greater than or equal to approximately 200 mm³. A plurality of the bulking devices can be implanted at spaced apart angular positions around the esophagus to form a ring-like, partial obstruction within the esophagus.

The invention may be embodied as a medical device for treatment of obesity, in which case the device may include an endoscopic delivery device sized for esophageal introduction into a patient, and a bulking device for implantation in a wall of the esophagus. A placement tool, deliverable via the endoscopic delivery device, implants the bulking device in the wall of the esophagus. As examples, the placement tool may take the form of a gripping member that grips the bulking device, such as a clamp or forceps that extends distally or laterally from the endoscopic delivery device. In other embodiments, the placement tool may be a needle sized to accommodate a bulking device for injection into the esophageal wall.

In comparison to known implementations of devices and method used for the treatment of obesity, various embodiments of the invention may provide one or more advantages. By partially obstructing the esophagus, the patient is incapable of consuming food at an excessive rate, and experiences discomfort during excessive food consumption. The partial obstruction physically limits excessive food consumption, while the discomfort provides a form of biofeedback that discourages the patient from excessive eating. The result is prevention of increased obesity and possibly weight loss. In this manner, the invention is capable of discouraging excessive consumption of food without the use of appetite suppressant medications, or chronic implantation of prostheses within the interior of the stomach. Also, in some embodiments, implantation of the bulking devices can be achieved endoscopically without the need for invasive surgical intervention or substantial modification of the stomach structure. Consequently, the invention can treat obesity with reduce side effects, reduced recovery time, and possible elimination of lengthy hospital stays.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a cross-sectional diagram of the interior of an esophagus and stomach with implanted bulking devices to form a partial obstruction of the esophagus.

FIG. 2 is a diagram illustrating deployment of an endoscopic delivery device shown in conjunction with an esophagus and stomach of a patient.

FIG. 3 is an enlarged view of a portion of the esophagus in the vicinity of the LES, illustrating preparation of an implantation pocket for a bulking device.

FIG. 4 is another enlarged view of a portion of the esophagus in the vicinity of the LES, illustrating injection of a fluid to prepare an implantation pocket.

FIG. 5 is another enlarged view of a portion of the esophagus in the vicinity of the LES, illustrating placement of a bulking device in an implantation pocket.

FIG. 6 is an enlarged view of a portion of the esophagus in the vicinity of the LES, illustrating expansion of bulking devices following implantation to produce a partial obstruction in the esophagus.

FIG. 7 is a cross-sectional end view of the esophagus taken across line A-A′ of FIG. 6, illustrating formation of a partial esophageal obstruction by a plurality of implanted bulking devices.

FIG. 8 is a diagram illustrating a method for implanting a bulking device in a wall of an esophagus to form a partial obstruction.

### DETAILED DESCRIPTION

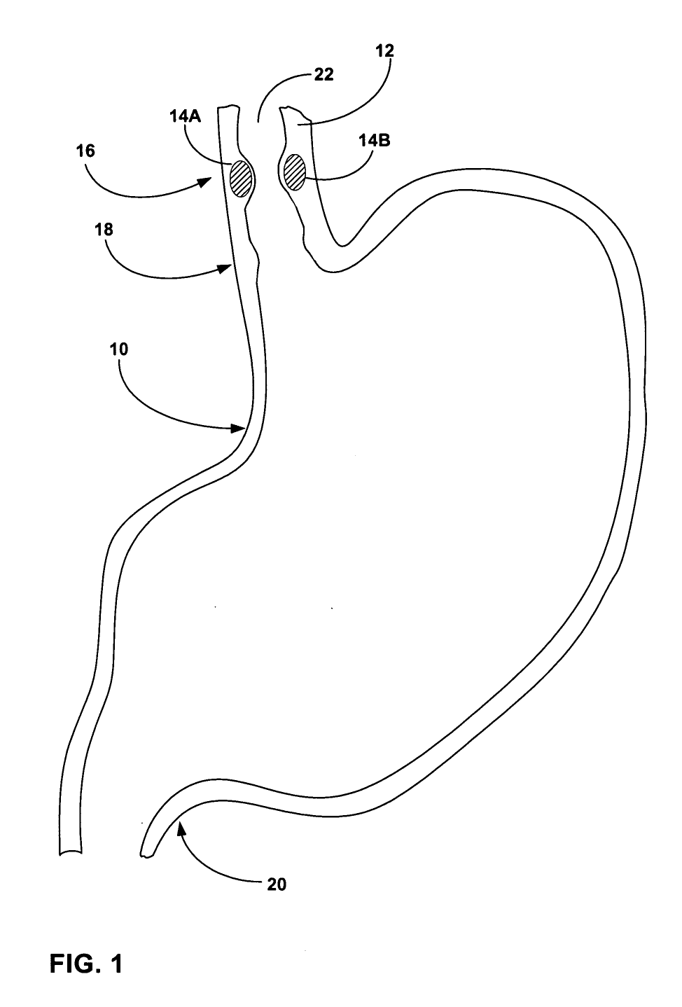

FIG. 1 is a cross-sectional diagram of the interior of a stomach 10 and esophagus 12 with implanted bulking devices 14A, 14B (hereinafter bulking devices 14) to form a partial obstruction 16 of the esophagus, in accordance with the invention. As shown in FIG. 1, esophagus 12 extends downward to join stomach 10 at lower esophageal sphincter (LES) 18, which regulates input to the stomach. Pyloric sphincter 20 joins stomach and the small intestine, and regulates output of stomach 10. Bulking devices 14 are implanted in a mucosal wall of esophagus 12 to reduce the diameter of an inner lumen 22 of the esophagus at a localized point and thereby form partial obstruction 16. Although FIG. 1 illustrates two bulking devices 14 in cross-section, additional bulking devices may be implanted at partial obstruction 16 at different angular positions about the circumference of esophagus 12.

Partial obstruction 16 in esophagus 12 serves to limit food intake by a patient, and thereby treat obesity. In particular, partial obstruction 16 is sized to permit passage of food, but at a reduced intake rate relative to an unobstructed esophagus 12. An inner lumen of a typical human esophagus has a diameter of approximately 2.5 to 3.5 cm. Partial obstruction 16 may be sized to reduce the effective diameter of inner lumen 22 to approximately 1.0 to 2.0 cm at the obstruction point.

With partial obstruction 16, the patient is incapable of consuming food at an excessive rate, and experiences discomfort during excessive food consumption. The partial obstruction physically limits excessive food consumption through inner lumen 22. At the same time, the discomfort may provide a form of biofeedback that discourages the patient from excessive eating. The result is prevention of increased obesity and weight loss.

Bulking devices 14 may be implanted surgically or endoscopically, although endoscopic implantation ordinarily will be desirable. In particular, endoscopic implantation of bulking devices 14 via esophagus 12 can prevent substantial trauma and recovery time otherwise associated with surgical implantation techniques. Endoscopic implantation may reduce the duration and complexity of the implantation procedure, and possibly eliminate the need for an overnight hospital stay in some instances. In addition, endoscopic implantation may be less likely to disrupt the physiological function of stomach 10, esophagus 12, and LES 18.

Bulking devices 14 may be formed from an expandable material that is initially implanted with a reduced, unexpanded size. Upon implantation, bulking devices 14 expand to a larger size to stretch the mucosal wall in esophagus 12 and create partial obstruction 16. As an example, bulking devices 14 may be a prosthesis formed from a hydrogel material that is implanted in an at least partially dehydrated state having a reduced size. Upon rehydration following implantation, bulking devices 14 assume an expanded state and increased size. Hence, the initial, unexpanded size of bulking devices 14 facilitates implantation, but subsequent expansion supports the formation of partial obstruction 16.

In the example of FIG. 1, bulking devices 14 have a substantially elliptical capsule-like shape. In other embodiments, bulking devices 14 may have a variety of shapes, e.g., substantially spherically shaped, rod- or cylinder-shaped, or irregularly shaped. In an at least partially dehydrated state for implantation, an elliptical capsule-shaped bulking device 14 may have a minor axis width of approximately 2 mm, and a major axis length of approximately 20 mm. Following implantation in mucosal wall 37, the capsule-shaped bulking device 14 may have a minor axis width of approximately 6 mm and a major axis length of approximately 15 mm. This corresponds to an exemplary pre-implantation volume of less than approximately 65 mm³ and a post-implantation volume of greater than or equal to approximately 400 mm³.

Hence, in general, in an at least partially dehydrated state for implantation, each bulking device 14 may have a volume of less than approximately 100 mm³. Upon expansion following implantation and subsequent rehydration, bulking device 14 may have a volume of greater than approximately 200 mm³. In some embodiments, each bulking device 14 may have a pre-implantation volume of less than or equal to approximately 75 mm³ and a post-implantation volume of greater than or equal to approximately 300 mm³. Hence, the hydrogel material may have an expansion ratio of greater than or equal to approximately 100 percent, or greater than or equal to approximately two times the pre-implantation size. In some embodiments, however, each bulking device 14 may have a larger volumetric expansion ratio, from an at least partially dehydrated state (pre-implantation) to a hydrated, expanded state (post-implantation), of at least approximately 4.5:1, and more particularly approximately 27:1. Other sizes and expansion ratios may be selected in accordance with the structural requirements for formation of a partial obstruction and the thickness of the esophageal wall in a given patient.

Bulking devices 14 may be placed at a series of regularly or irregularly spaced angular positions about the circumference of inner lumen 22 of esophagus 12. In some embodiments, two, three, four or more bulking devices 14 may be placed in esophagus 12. Spacing between adjacent bulking devices 14 may be controlled by taking into account the expanded size of the bulking devices. Adjacent bulking devices 20 may be separated about the circumference of inner lumen 22 by a section of intact mucosal tissue within the wall of esophagus 12. By leaving a substantial portion of the mucosal tissue intact, bulking devices 14 can contribute to partial obstruction 16 without substantially compromising the physiological function of esophagus in the digestion process.

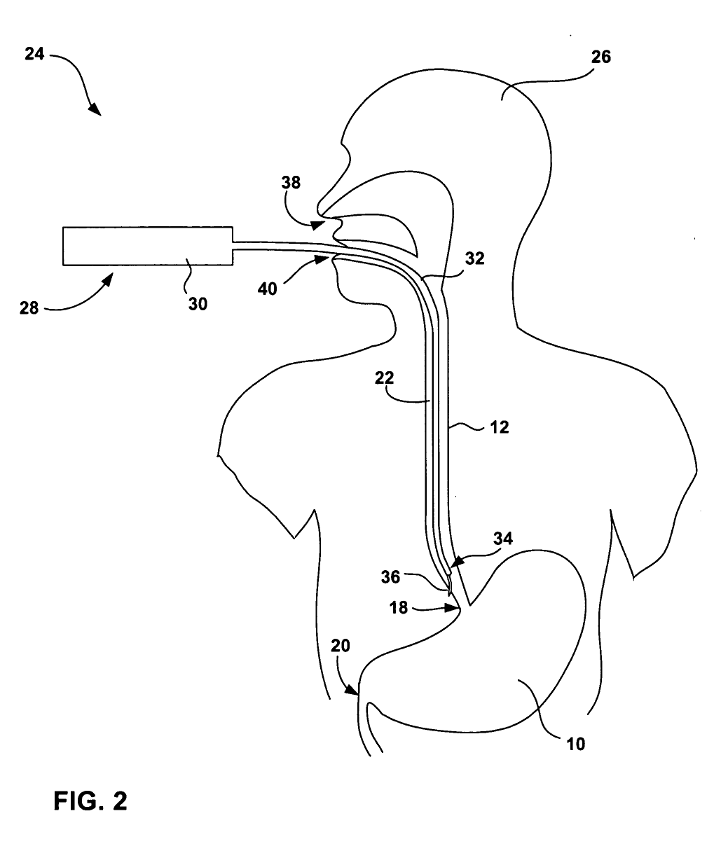

FIG. 2 is a diagram illustrating deployment of an endoscopic delivery system 24 shown in conjunction with a stomach 10 of a patient 26. As shown in FIG. 2, esophageal delivery system 24 serves to position and place bulking devices 14 within esophagus 12 of patient 26. Esophageal delivery device 24 includes an endoscopic delivery device 28 having a proximal portion, referred to herein as a handle 30, and a flexible probe 32 that extends from handle 30 into the gastrointestinal tract of patient 26.

A bulking device 14 is delivered to a target location in the mucosal wall of esophagus 12 via a distal end 34 of flexible probe 32. In particular, one or more placement tools 36 may extend distally or laterally from distal end 34 of flexible probe for preparation of an implantation pocket and implantation of a bulking device 14. Distal end 34 of delivery device 28 enters esophagus 12, via either nasal cavity 38 or oral cavity 40, and extends into esophagus 10 to a desired placement location above LES 18. Upon implantation of a bulking device 14, endoscopic delivery device 28 may be repositioned within esophagus 12 to implant other bulking devices.

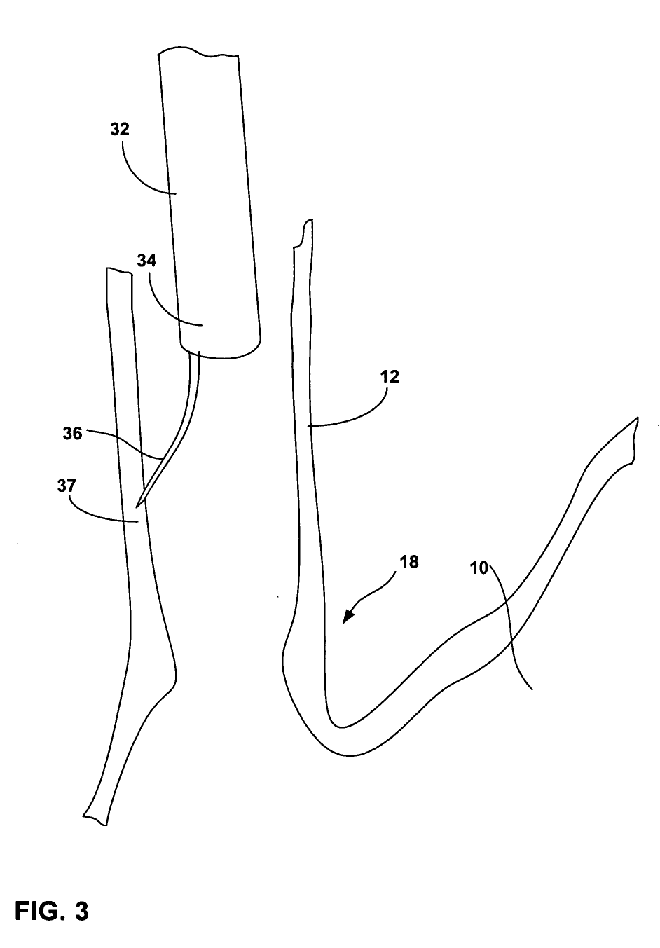

FIG. 3 is an enlarged view of a portion of the esophagus 12 in the vicinity of the LES 18, illustrating optional preparation of an implantation pocket for a bulking device 14. Formation of an implantation pocket prior to implantation of bulking device 14 may be not be necessary. As will be described, however, the formation of an implantation pocket may be advantageous in that it serves to stretch and define an area in the mucosal wall of esophagus 12 to provide space to receive the bulking device 14, and perhaps accommodate some of the expansion of the bulking device.

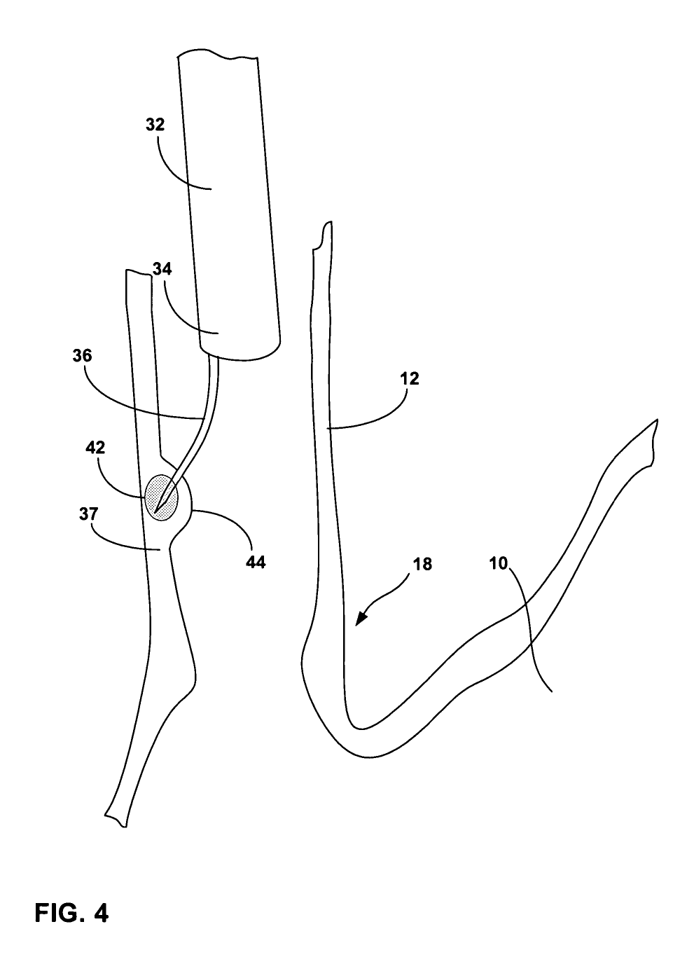

FIG. 4 is another enlarged view of a portion of the esophagus 12 in the vicinity of the LES 18, illustrating injection of a fluid 42 to prepare an implantation pocket 44. Formation of implantation pocket 44 may be optional, and may not be necessary in some embodiments. As shown in FIGS. 3 and 4, a physician extends a needle 36 along the length of flexible probe 32 and out from distal end 34 to penetrate mucosal wall 37 of esophagus 12. For example, the physician may steer distal end 34 of flexible probe 32 to a desired location on mucosal wall 37 using conventional endoscopic steering equipment, such as embedded pre-formed wires or the like. Upon penetration of mucosal wall 37, the physician injects a bolus 42 of saline or other biocompatible fluid to expand a localized region of the mucosal wall and create an implantation pocket 44.

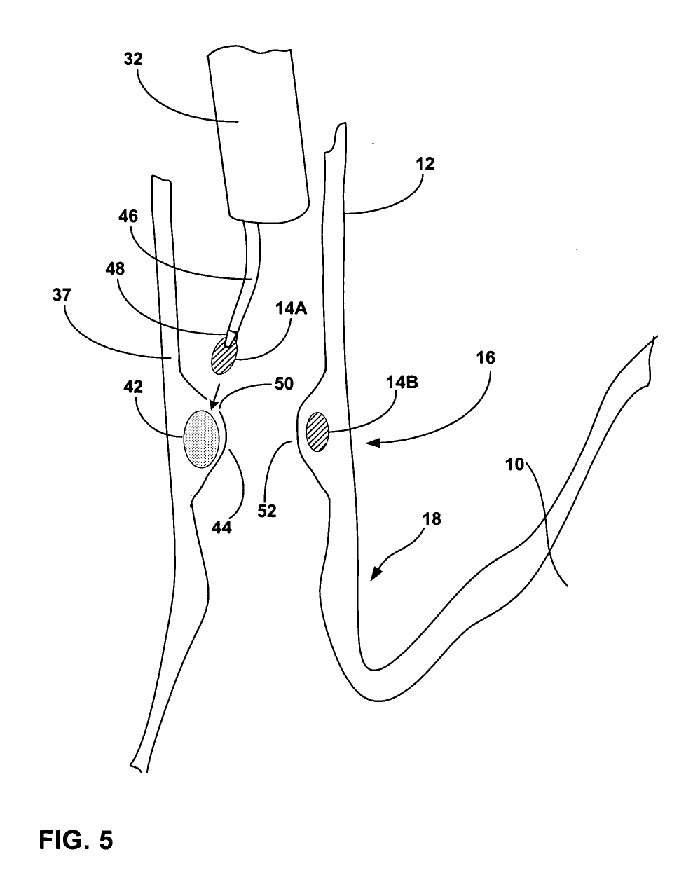

FIG. 5 is another enlarged view of a portion of the esophagus 12 in the vicinity of the LES 18, illustrating placement of a bulking device 14A in implantation pocket 44. In FIG. 5, bulking device 14B is shown following implantation in implantation pocket 52. Bulking device 14A is shown in the process of implantation. In particular, bulking device 14A is implanted into implantation pocket 44 via an aperture 50 formed in mucosal wall 37. Aperture 50 may be formed by a cutting tool or blunt dissection. A placement tool 46 extends along the length of flexible probe 32 and protrudes from a distal end of the flexible probe. Placement tool 46 may be independently steerable to locate the desired implantation pocket 44.

In the example of FIG. 5, placement tool 46 includes a gripping member, such as a pair of jaws or forceps, that carry bulking device 14A for placement within implantation pocket 44 via aperture 50. Upon placement of bulking device 14A within implantation pocket 44, a suture or stapling tool may be introduced via flexible probe 32 to close aperture 50. Precise positioning may be aided by endoscopic viewing provided by an imaging endoscope integrated within or delivered simultaneously with flexible probe 32. In addition, external imaging techniques such as fluoroscopy or ultrasonic imaging may be used to aid precise positioning.

As an alternative (not shown) to implantation with a gripping member, in some embodiments, a bulking device 14 may be initially mounted in a tip of a needle, which may be introduced via flexible probe 32. Upon placement of the tip of needle within the esophageal wall at a desired implantation site, a physician expels bulking device 14 from the needle. The physician may actuate a fluid pressure source or elongated push rod to drive bulking device 14 out of the needle and into the esophageal wall. Following implantation via the needle, bulking device expands, e.g., by rehydration, to assume an enlarged size. Then, the needle and flexible probe 32 may be withdrawn or repositioned to implant another bulking device 14 at a different tissue site within the esophageal wall. As an example, the needle may have a diameter in the range of less than approximately 2 mm to 4 mm in inside diameter, which can accommodate a spherical or rod-like bulking device 14 having a diameter or transverse cross-section, respectively, of approximately 1.5 mm to 3.5 mm in diameter. Upon implantation of bulking device 14 with a needle, the implantation hole may be sufficiently small that there is not a need for suturing or stapling.

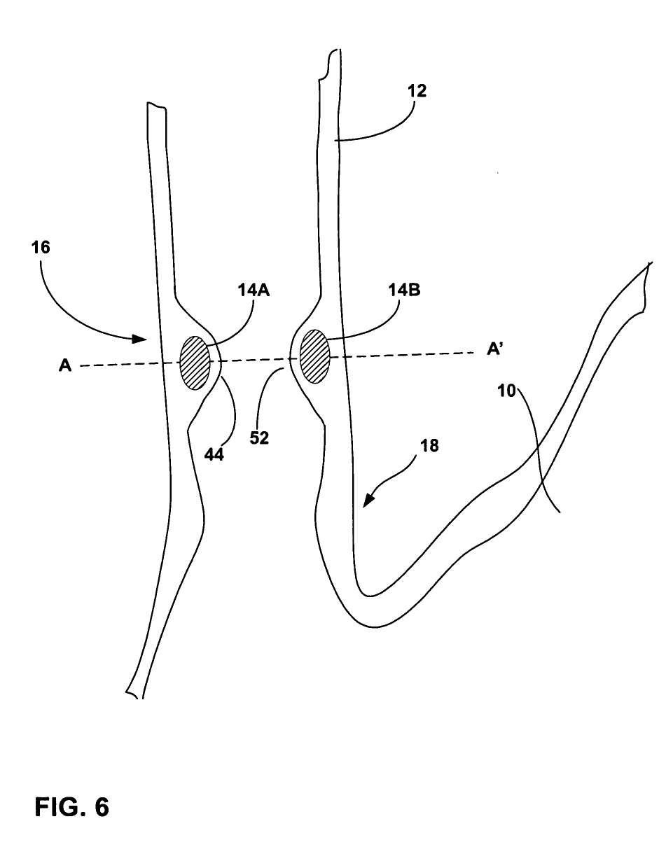

FIG. 6 is an enlarged view of a portion of the esophagus 12 in the vicinity of the LES 18, illustrating expansion of bulking devices 14 following implantation to produce a partial obstruction 16 in the esophagus. In FIG. 5, bulking devices 14 are shown at the time of implantation in an initial, unexpanded state. In FIG. 6, however, bulking devices 14 are shown following implantation in an expanded state. As discussed above, bulking devices 14 may be formed from a variety of expandable materials that permit implantation of the bulking devices in an initial, reduced size, followed by post-implant expansion to form partial obstruction 16 of esophagus 12.

As an example, bulking devices 14 may be formed from a hydrogel material that is implanted in an at least partially dehydrated state. In a dehydrated state, the hydrogel materials is reduced in size. Following implantation within mucosal wall 37, bulking device 14 takes on moisture and rehydrates. In this manner, bulking device 14 expands to an enlarged size that further increases the size of partial obstruction 16. A hydrogel material may be implanted as a solid prosthesis, as shown in FIGS. 3-6, or injected as a fluid material that becomes solid or semi-solid following injection.

Partial obstruction 16 is formed within esophagus 12 at any desired position between LES 18 and the upper esophageal sphincter (UES) (not shown) of the patient. The length of the esophagus varies from patient to patient, but is on the order or approximately 25 cm. As one example, partial obstruction 16 may be formed approximately midway between LES and the UES, e.g., approximately 8 to 16 cm above the LES. In other embodiments, it is sufficient that partial obstruction 16 be formed a short distance above the LES, e.g., greater than or equal to approximately 2 cm above the LES. In either case, partial obstruction 16 is not placed extremely close to either LES or UES, and therefore is less likely to alter or impair the function of either LES or UES. In other embodiments, however, a physician may elect to place partial obstruction 16 more closely to LES 18 or the UES.

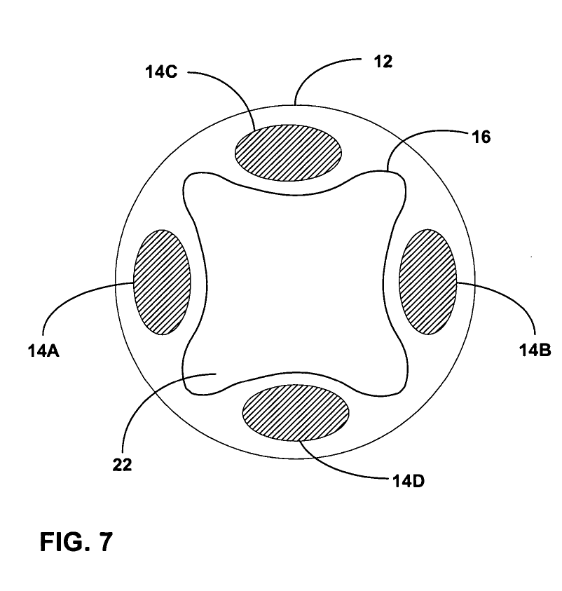

FIG. 7 is a cross-sectional end view of the esophagus 12 taken across line A-A′ of FIG. 6, illustrating formation of a partial esophageal obstruction by a plurality of implanted bulking devices 14A, 14B visible in FIG. 6 and additional bulking devices 14C, 14D. In the example of FIG. 7, individual bulking devices 14A-14D are implanted at angular positions spaced approximately 90 degrees apart from one another around esophagus 12. In this manner, bulking devices 14A-14D combine to produce a partial obstruction 16 of the inner lumen 22 of esophagus 12. The number of bulking devices 14 implanted in esophagus 12 may vary, and may be more or less than the number of bulking devices shown in the example of FIG. 7.

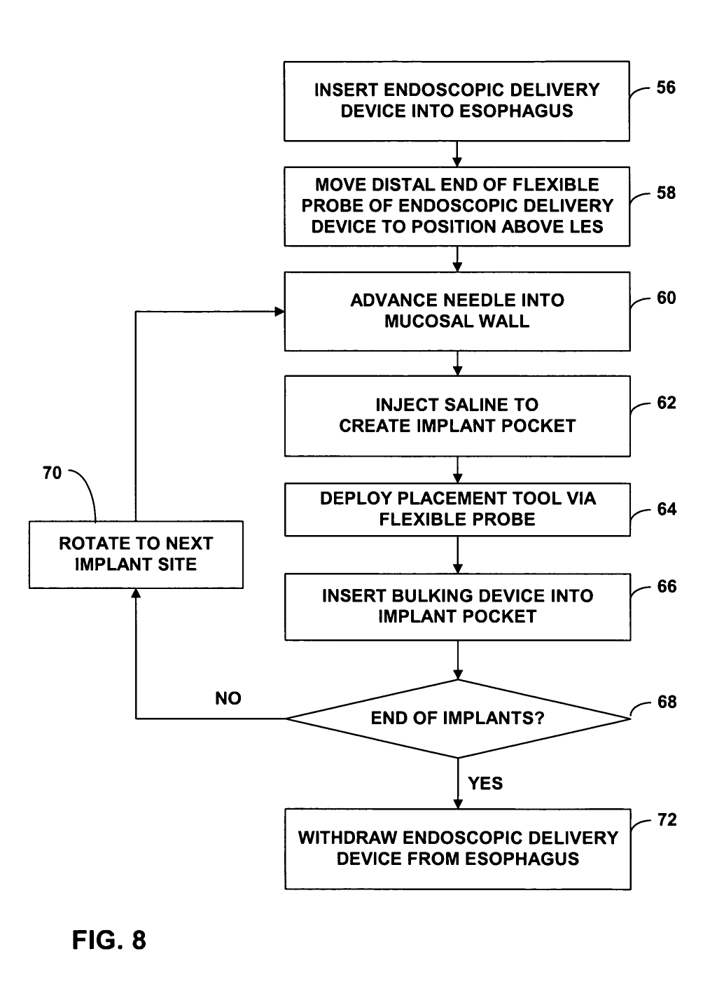

FIG. 8 is a diagram illustrating a method for implanting a bulking device 14 in a mucosal wall 37 of an esophagus 12 to form a partial obstruction. As shown in FIG. 8, a physician inserts an endoscopic delivery device into the esophagus of a patient (56), and moves a distal end of a flexible probe to a position above the LES (58). The physician then advances a needle from the distal end of the flexible probe and into a mucosal wall in the esophagus above the LES (60). Once the distal tip of the needle is in place, the physician injects saline or another fluid into the mucosal wall to create an implantation pocket (62).

The physician then withdraws the needle, and deploys a placement tool via the flexible probe (64), and implants the bulking device into the implant pocket (66). The placement tool may take the form of a needle or gripping device. If additional bulking devices are to be implanted (68), the physician repositions the flexible probe to another implant site (70) in the esophagus and repeats the implantation process. When all bulking devices have been implanted, the physician withdraws the endoscopic delivery device from the esophagus (72).

A bulking device 14, as described herein, preferably is soft and compliant to minimized trauma within mucosal layer 37 upon implantation. The bulking device may be constructed from a variety of biocompatible polymeric materials. Again, the materials forming bulking device may be expandable. In particular, as described herein, the bulking devices may be formed from an expandable hydrogel material. Suitable materials, including hydrogel materials, are described in U.S. Pat. No. 6,401,718 to Johnson et al., assigned to Medtronic Endonetics, Inc., and entitled “Submucosal esophageal bulking device,” the entire content of which is incorporated herein by reference.

As alternatives, described in Johnson et al., bulking device 14 may take the form of a fluid-filled, flexible capsule, pillow or balloon made from elastomeric materials such as silicone, latex, urethane, and the like. Example fillers include biocompatible liquid or gel such as saline, silicone oil, DMSO, polyvinyl, pyrollidone and hydrogels. As a further alternative, the bulking device may be a unitary structure formed by molding, casting, stamping or the like. The unitary structure may formed from hydrogel material, biocompatible foam material such as silicone foam or polyurethane foam, or a variety of biocompatible materials such as silicone, polyurethane, polysulfone, polyester, and the like. As described in Johnson et al., foam material may include outer skin of porous foam that facilitates tissue ingrowth.

As alternatives to implanted solid materials, bulking devices may be formed by injected fluids that form solids following injection. A variety of implanted solid materials and injected fluids suitable for formation of bulking devices form a partial obstruction of the esophagus, as described herein, are disclosed in U.S. Published Patent Application No. 20040019388, to Starkebaum, assigned to Medtronic, Inc. and entitled “Methods and implants for retarding stomach emptying to treat eating disorders,” the entire content of which is incorporated herein by reference. Accordingly, bulking devices may refer to solid, semi-solid, or filled implants, or injected fluids that formed solid or semi-solid bulking devices within mucosal wall 37 of esophagus 12 to treat obesity.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems as described herein.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A method for treatment of obesity, the method comprising implanting one or more bulking devices in a wall of an esophagus of a patient at a position above a lower esophageal sphincter within the esophagus, wherein the implanted bulking devices are sized to expand the mucosal wall to an extent sufficient to create a partial occlusion of an inner lumen of the esophagus.

2. The method of claim 1, further comprising implanting the bulking devices at a distance of greater than or equal to approximately 2 cm above the lower esophageal sphincter.

3. The method of claim 1, further comprising endoscopically implanting the bulking devices within the esophagus of the patient.

4. The method of claim 1, further comprising repositioning an endoscopic device among a plurality of implantation sites to endoscopically implant a plurality of the bulking devices.

5. The method of claim 1, wherein each of the bulking devices includes a solid, hydrogel material.

6. The method of claim 5, further comprising implanting the bulking devices with the hydrogel material in an at least partially dehydrated state, the bulking devices rehydrating upon implantation and thereby expanding in size to expand the mucosal wall.

7. The method of claim 1, wherein the bulking devices are expandable following implantation.

8. The method of claim 7, wherein the bulking devices are expandable from a pre-implantation volume of less than approximately 100 mm³ to a post-implantation size of greater than or equal to approximately 200 mm³.

9. The method of claim 8, wherein the bulking devices are expandable from a pre-implantation size to a post-implantation size of greater than or equal to approximately two times the pre-implantation size.

10. The method of claim 1, wherein each of the bulking devices, following implantation, has a volume of greater than or equal to approximately 200 mm³.

11. The method of claim 1, wherein each of the bulking devices is substantially capsule-like in shape, substantially spherical in shape substantially disc-like in shape, or substantially tubular in shape.

12. The method of claim 1, further comprising implanting a plurality of the bulking devices at different angular positions about the inner lumen of the esophagus.

13. The method of claim 12, wherein the bulking devices include four bulking devices spaced at approximately 90 degree intervals from one another.

14. The method of claim 1, further comprising injecting a fluid into the mucosal wall to create an implantation pocket, and implanting one of the bulking devices in the implantation pocket.

15. A system for treatment of obesity, the system comprising:
an endoscopic delivery device sized for introduction into an esophagus of a patient;
a bulking device for implantation in a mucosal wall of the esophagus, wherein the implanted bulking devices are sized to expand the mucosal wall to an extent sufficient to create a partial occlusion of an inner lumen of the esophagus; and
a placement tool, deliverable via the endoscopic delivery device, to implant the bulking device in the mucosal wall of the esophagus at a position above a lower esophageal sphincter of the esophagus.

16. The system of claim 15, wherein the bulking device includes a solid, hydrogel material.

17. The system of claim 15, wherein the solid, hydrogel material is in an at least partially dehydrated state prior to implantation, and the bulking devices rehydrates upon implantation and thereby expands in size to stretch the mucosal wall.

18. The system of claim 15, wherein the bulking device is expandable from a pre-implantation volume of less than approximately 100 mm³ to a post-implantation size of greater than or equal to approximately 200 mm³.

19. The system of claim 15, wherein the bulking device is expandable from a pre-implantation size to a post-implantation size of greater than or equal to approximately two times the pre-implantation size.

20. The system of claim 15, wherein the bulking device, following implantation, has a volume of greater than or equal to approximately 200 mm³.

21. The system of claim 15, wherein each of the bulking devices is substantially capsule-like in shape, substantially spherical in shape substantially disc-like in shape, or substantially tubular in shape.

22. The system of claim 15, wherein the placement tool is repositionable to implant a plurality of the bulking devices at different angular positions about the inner lumen of the esophagus.

23. The system of claim 15, wherein the bulking devices include four bulking devices for implantation at intervals spaced approximately 90 degree apart from one another.

24. The system of claim 15, further comprising a needle deliverable via the endoscopic delivery device to inject a fluid into the mucosal wall to create an implantation pocket for the bulking device.

25. The system of claim 15 further comprising a needle, deliverable via the endoscopic delivery device, to inject a fluid into the mucosal wall to create an implantation pocket for the bulking device.

26. A system for treatment of obesity, the system comprising:
an endoscopic delivery device sized for introduction into an esophagus of a patient;
means, implantable in a mucosal wall of the esophagus, for expanding the mucosal wall to an extent sufficient to create a partial occlusion of an inner lumen of the esophagus; and
means for implanting the bulking device in the mucosal wall of the patient via the endoscopic delivery device at a position above a lower esophageal sphincter of the esophagus.

27. The system of claim 26, wherein the expanding means includes a solid, hydrogel material implantable within the mucosal wall of the esophagus.

28. The system of claim 26, wherein the hydrogel material is in an at least partially dehydrated state prior to implantation, and rehydrates upon implantation to thereby expand in size to create the partial occlusion.

29. The system of claim 26, wherein the expanding means is expandable from a pre-implantation volume of less than approximately 100 mm³ to a post-implantation size of greater than or equal to approximately 200 mm³.

30. The system of claim 26, wherein the expanding means is expandable from a pre-implantation size to a post-implantation size of greater than or equal to approximately two times the pre-implantation size.

31. The system of claim 26, wherein the expanding means, following implantation, has a volume of greater than or equal to approximately 200 mm³.

32. A system for treatment of obesity, the system comprising:
an endoscopic delivery device sized for esophageal introduction into an esophagus of a patient;
a solid, expandable hydrogel prosthesis, implantable in a mucosal wall of the patient, to expand the mucosal wall to an extent sufficient to create a partial occlusion of an inner lumen of the esophagus; and
an implant tool, deliverable via the endoscopic delivery device, to implant the bulking device in the mucosal wall at a position above a lower esophageal sphincter of the esophagus via the endoscopic delivery device.

33. The system of claim 32, wherein the hydrogel prosthesis is in an at least partially dehydrated state prior to implantation, and the hydrogel prosthesis rehydrates upon implantation and thereby expands in size to stretch the mucosal wall.

34. The system of claim 32, wherein the hydrogel prosthesis is expandable from a pre-implantation volume of less than approximately 100 mm³ to a post-implantation size of greater than or equal to approximately 200 mm³.

35. The system of claim 32, wherein the hydrogel prosthesis is expandable from a pre-implantation size to a post-implantation size of greater than or equal to approximately two times the pre-implantation size.

36. The system of claim 32, wherein the hydrogel prosthesis, following implantation, has a volume of greater than or equal to approximately 200 mm³.

37. The system of claim 32, wherein the hydrogel prosthesis is substantially capsule-like in shape, substantially spherical in shape substantially disc-like in shape, or substantially tubular in shape.
